(19) United States
(12) Patent Application Publication
(10) Pub. No.: US 2008/0140003 A1
(43) Pub. Date: Jun. 12, 2008

(54) BALLOON CATHETER HAVING A REGROOMING SHEATH AND METHOD FOR COLLAPSING AN EXPANDED MEDICAL DEVICE

(75) Inventors: Nianjiong J. Bei, Foster City, CA (US); Danielle Laflash, San Mateo, CA (US); Patrick C. Saxton, Santa Clara, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ADVANCED CARDIOVASCULAR SYSTEMS, INC., SANTA CLARA, CA (US)

(21) Appl. No.: 11/567,647

(22) Filed: Dec. 6, 2006

(51) Int. Cl. A61M 25/10

(52) U.S. Cl. 604/103.05

(57) ABSTRACT

A method of using a balloon catheter to perform a medical procedure at a treatment site in a patient's body lumen and to recover an expanded device, such as an embolic protection device, which is adjacent to the treatment site in the body lumen. The inflated balloon is deflated and regroomed to a low profile configuration in the body lumen, and the balloon catheter is advanced distally from the treatment site to collapse the expanded device (e.g., embolic protection filter) within the balloon catheter. The balloon catheter has a recovery distal tip for collapsing an expanded device, and has a regrooming sheath member configured to slidably receive the deflated balloon therein to regroom the balloon.

### BACKGROUND OF THE INVENTION

The present invention relates generally to interventional catheters, and more particularly to a balloon catheter configured for use in an intravascular medical procedure in a stenosed blood vessel.

The treatment of an occluded region of a patient's vasculature commonly includes a percutaneous transluminal interventional procedure such as inflating a catheter balloon and/or implanting a stent inside the blood vessel at the site of the stenosis. For example, in balloon angioplasty, the catheter balloon is positioned across the lesion and inflated with fluid one or more times to a predetermined size at relatively high pressures (e.g. greater than 8 atmospheres) so that the stenosis is compressed against the arterial wall and the wall expanded to clear the passageway. Physicians frequently implant a stent inside the blood vessel at the site of the lesion. Stents may also be used to repair vessels having an intimal flap or dissection or to generally strengthen a weakened section of a vessel. Stents are usually delivered to a desired location within the blood vessel in a contracted condition, and expanded to a larger diameter by release of a radially restraining force (for self-expanding stents) and/or by expansion of a balloon (for balloon expandable stents). The delivery catheter is withdrawn and the expanded stent left implanted within the blood vessel at the site of the dilated lesion.

Such intravascular procedures may release emboli into the circulatory system, which can be extremely dangerous to the patient. Debris that is carried by the bloodstream to distal vessels of the brain may cause these cerebral vessels to occlude, resulting in a stroke, and in some cases, death. Thus, when performed in a carotid artery, an embolic protection device to capture and collect released emboli may be deployed downstream to the interventional catheter. For example, embolic protection devices in the form of filters or traps can be delivered in a collapsed configuration to a location adjacent to the interventional procedure site, radially expanded to open the mouth of the filter or trap, and after the interventional procedure has been performed, the device is collapsed for removal with the captured embolic material therein.

An essential step in effectively performing an interventional procedure is properly positioning the catheter system at a desired location within the patient's vasculature. The catheter shaft must be able to transmit force along the length of the catheter shaft to allow it to be pushed through the vasculature. However, the catheter shaft must also retain sufficient flexibility and low profile to allow it to track over a guidewire through the often tortuous, narrow vasculature. Such deliverability issues must be balanced against one another and against other performance characteristics. As a result, one design challenge has been making the procedure, including the delivery and retrieval of the components of the catheter system, as quick and easy as possible.

### SUMMARY OF THE INVENTION

The invention is directed to a balloon catheter, and a method of using a balloon catheter to perform a medical procedure at a treatment site in a patient's body lumen and to recover an expanded device, such as an embolic protection device, which is adjacent to the treatment site in the body lumen. The method generally comprises inflating a catheter balloon in the patient's body lumen at the interventional treatment site, deflating the balloon, regrooming the deflated balloon to a low profile configuration, and advancing the regroomed balloon catheter distally from the treatment site in order to collapse the expanded device (e.g., embolic protection filter) within a lumen of the balloon catheter. The balloon catheter containing the expandable device in the collapsed configuration can then be withdrawn from the vessel, with the balloon in the regroomed configuration having a low profile which facilitates this withdrawal. A balloon catheter of the invention has a recovery distal tip configured for collapsing an expanded device (e.g., embolic protection filter), and has a regrooming sheath member configured to slidably receive the deflated balloon therein to regroom the balloon to a low profile configuration.

In one embodiment, the method of the invention comprises advancing within a patient's body lumen a balloon catheter having an elongated shaft with an inflation lumen extending therein, an inflatable balloon secured to a distal shaft section such that an interior of the balloon is in fluid communication with the inflation lumen, a distal tip configured to slidably receive a frame or other structure of an expanded device such as an embolic protection device to collapse the frame, and a regrooming sheath releasably locked on the elongated shaft and slidably disposed thereto in an unlocked configuration. The method includes inflating the balloon in the patient's body lumen at a treatment location longitudinally adjacent to the expanded frame to perform a medical procedure. The inflated balloon is then deflated, and the method includes regrooming the deflated balloon by slidably displacing the regrooming sheath and balloon relative to one another to position the balloon within the regrooming sheath to reduce the profile of the deflated balloon to a regroomed configuration. The expanded frame is then collapsed by being positioned within the balloon catheter distal tip, preferably by sliding the balloon catheter out the distal end of the regrooming sheath and advancing the distal tip of the balloon catheter over at least a portion of the expanded frame. As a result, the balloon in the regroomed configuration is advanced to a location distal to the regrooming sheath. The balloon catheter with the collapsed frame therein can then be slidably displaced in the patient's body lumen to reposition or remove the frame from the body lumen.

In one embodiment, the balloon is inflated to radially expand a stent at the treatment site in the body lumen. Typically, the stent has been previously delivered and deployed at the treatment site using a separate stent delivery catheter prior to the advancement and inflation of the balloon catheter of the invention, in which case the balloon is being inflated to insure that the stent is in a fully expanded configuration by further expanding the partially expanded stent (commonly referred to as post-dilation, or stent touch-up). For example, self-expanding stents which are held in a collapsed configuration for delivery to the treatment site by the radially restraining force of a stent sheath therearound, and which radially expand upon retraction of the stent sheath, typically require a post-dilation to fully expand the stent against the vessel wall. Details regarding self-expanding stents and delivery systems can be found in U.S. Pat. Nos. 6,695,862 and 6,582,460 incorporated by reference herein in their entireties. However, in the method of the invention, the balloon can be inflated for a variety of alternative procedures including deploying a stent delivered to the treatment site on the balloon, dilatation of the vessel, drug delivery, and the like.

The balloon catheter has a distal tip configured to recover an embolic protection device or other expandable device (i.e., a device which reversibly radially expands and collapses). Although discussed below primarily in terms of the embodiment in which the balloon catheter distal tip is used to recover an embolic protection device, it should be understood that the distal tip can be configured for recovering a variety of deployed devices which are recovered by radially collapsing from an expanded configuration to a collapsed configuration as the recovery tip is slidably positioned around the device in a patient's body lumen. Thus, the expandable device can be configured to be deployed and then retrieved within a body lumen for a variety of purposes including, for example, drug or fluid delivery, and temporary support of the body lumen.

In a presently preferred embodiment, the distal tip has a length sufficiently long to collapse the embolic protection device therein and permit withdrawal of the collapsed device proximally through the implanted stent, without requiring a separate outer recovery catheter to be advanced over the distal tip and the distal end of the embolic protection device. As a result, the system of the invention has a low profile which facilitates maneuvering and withdrawing the system within the body lumen. However, the distal tip length is sufficiently short to avoid it prematurely collapsing the embolic protection device during the interventional procedure. The distal tip has an inner diameter configured to slidably receive and thereby collapse the embolic protection device therein, and a wall thickness/strength which is sufficient to contain the collapsed device therein without any structural failure of the distal tip wall. However, the outer diameter and wall thickness of the distal tip is preferably minimized in order to provide the distal tip with sufficient flexibility and low profile to facilitate tracking and advancing the catheter into distal tortuous anatomy.

In accordance with the invention, the deflated balloon is regroomed prior to the embolic protection device being recovered within the distal tip of the balloon catheter. Specifically, after being inflated, the balloon forms wrinkles or folds of excess material upon being deflated which can result in the balloon snagging on an implanted prosthesis (e.g., stent) in the body lumen or releasing embolic particulates as the deflated balloon is slid within the body lumen. For example, if the deflated balloon were to snag on the stent edge, the retracting force of the balloon catheter might dislodge the stent, or tear the balloon leaving a balloon fragment behind in the body lumen, or cause a detachment or separation of catheter components bonded together. In contrast, the regroomed balloon according to an embodiment of the invention has the wrinkles or wings of excess deflated material wrapped around the balloon or otherwise smoothed to a lower profile than prior to being regroomed. As a result, during withdrawal of the regroomed balloon catheter, the highly disadvantageous and dangerous interactions with the stent or vessel anatomy are prevented or eliminated.

The regrooming sheath is preferably an elongated tube, sized and configured to minimize its affect on the profile and trackability of the balloon catheter. As a result, in a presently preferred embodiment, the regrooming sheath has an inner diameter along at least a distal section which is less than the outer diameter of the recovery distal tip of the balloon catheter. The inner diameter of at least a distal section of the regrooming sheath is sufficiently small to fit tightly onto the deflated balloon, to effectively regroom the balloon.

During retrieval of an embolic protection device following an intravascular procedure the balloon catheter of the invention minimizes procedure time and difficulty by avoiding the need to withdraw the balloon catheter and advance a separate recovery catheter within the body lumen. However, the balloon catheter of the invention is configured to be highly trackable and low profile, and to prevent or inhibit disadvantageous balloon interactions in stented and/or nonstented regions of the patient's vasculature during withdrawal of the balloon catheter therefrom. These and other advantages of the invention will become more apparent from the following detailed description and accompanying exemplary drawings.

### BRIEF DESCRIPTION OF THE DRAWINGS

FIG. 1 is an elevational, partially in section, view of a balloon catheter embodying features of the invention.

FIG. 2 is a transverse cross section of the catheter of FIG. 1, taken along line 2-2.

FIG. 3 is an enlarged longitudinal cross sectional view of the distal end of the balloon catheter of FIG. 1, taken within circle 3.

FIG. 4 is a transverse cross section of the catheter of FIG. 3, taken along line 4-4.

FIGS. 5-9 illustrate the balloon catheter of FIG. 1 in a method of performing a procedure embodying features of the invention, with FIG. 5 illustrating the balloon catheter noninflated balloon positioned at a treatment site in a patient's body lumen and proximal to a deployed embolic protection device.

FIG. 6 illustrates the balloon catheter of FIG. 5 with the balloon inflated to radially expand a stent.

FIG. 7 illustrates the balloon catheter of FIG. 6 with the balloon deflated prior to being regroomed.

FIG. 8 illustrates the balloon catheter of FIG. 7 with the balloon positioned within the regrooming sheath during regrooming.

FIG. 9 illustrates the balloon catheter of FIG. 8 with the embolic protection device radially collapsed in the balloon catheter device lumen, and with the balloon in a regroomed configuration.

FIG. 10 illustrates a rapid exchange-type balloon catheter embodying features of the invention, having a balloon protective cover on the balloon.

FIG. 11 is a perspective view of a distal portion of the balloon catheter of FIG. 10 during removal of the balloon protective cover from the balloon.

### DETAILED DESCRIPTION OF THE PREFERRED EMBODIMENTS

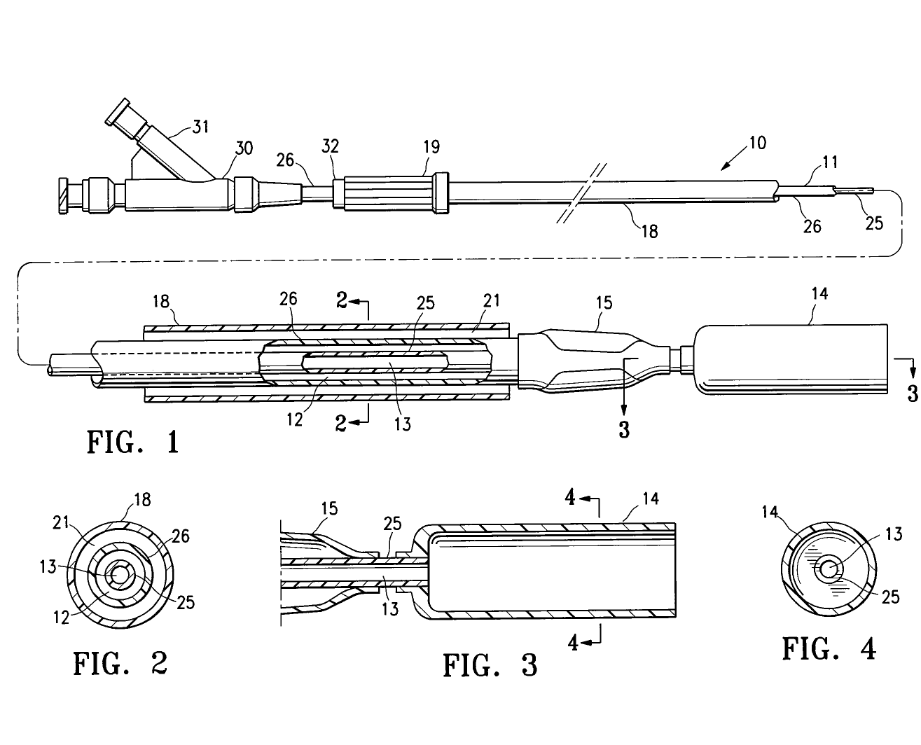

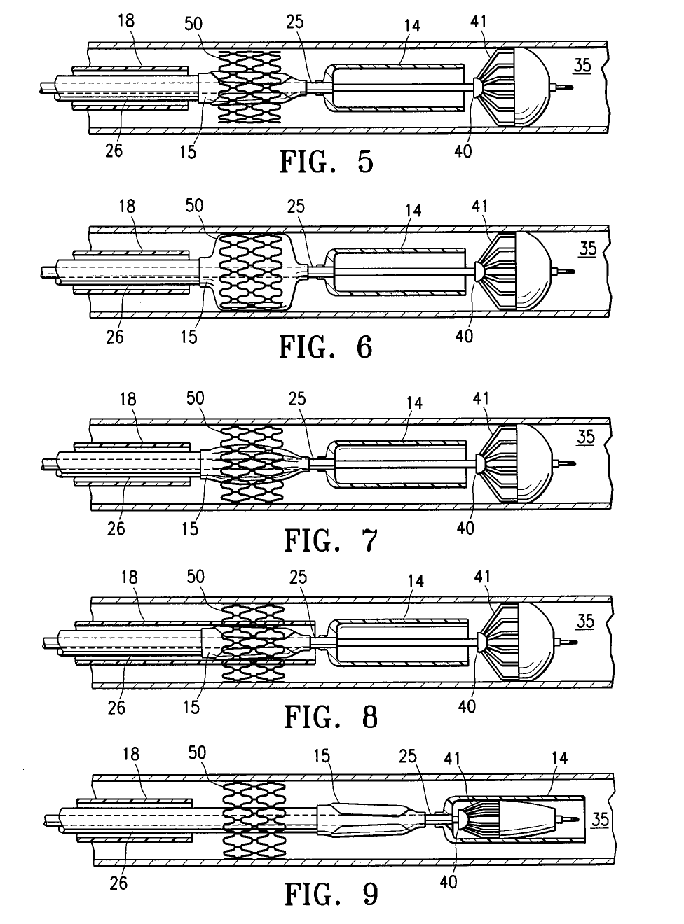

FIG. 1 illustrates an elevational, partially in section, view of a balloon catheter 10 embodying features of the invention, generally comprising an elongated catheter shaft 11 with a proximal end, a distal end, an inflation lumen 12, a device lumen 13, and a distal tip 14 configured to slidably receive at least a portion of an expanded section of an expandable device, such as an embolic protection device 40 (see FIG. 5), to collapse the expanded section to a collapsed configuration. The balloon catheter 10 has an inflatable balloon 15 on a distal shaft section with an interior in fluid communication with the inflation lumen 12, such that the balloon can be inflated from a noninflated configuration to an inflated configuration upon the introduction of inflation fluid to the balloon interior, and deflates to a deflated configuration upon the withdrawal of the inflation fluid. FIG. 1 illustrates the balloon 15 in the low profile noninflated configuration, which typically has wings of balloon material tightly wrapped around the balloon, for introduction and advancement within the patient's body lumen prior to inflation of the balloon 15. An adapter 30 on the proximal end of the catheter 10 provides access to the device lumen 13, and has a side arm 31 which is in fluid communication with the inflation lumen 12 and which is configured for connecting to an inflation fluid source (not shown).

The balloon catheter 10 has a regrooming sheath 18 releasably locked on the elongated shaft 11, and slidably disposed thereto in an unlocked configuration such that the regrooming sheath 18 can be slidably advanced over the deflated balloon 15 following deflation of the inflated balloon to regroom the deflated balloon 15 to a low profile configuration. Specifically, the regrooming sheath 18 comprises an elongated tube preferably having a single lumen 21, the sheath lumen 21 having at least a section which has an inner diameter less than a transverse dimension of the deflated balloon 15 and which is configured to slidably receive the deflated balloon 15 therein to reduce the profile of the deflated balloon 15 to a regroomed configuration. In the retracted configuration the sheath 18 distal end is proximal to the balloon, and in the advanced configuration the sheath 18 is configured to extend to the distal end of the balloon 15 (see FIG. 8). In a presently preferred embodiment, the regrooming sheath 18 has a length less than the balloon catheter shaft 11, such that in a fully retracted configuration the distal end of the regrooming sheath is proximal to the inflatable section of the balloon, and the proximal end of the regrooming sheath 18 is distal to the proximal end of the balloon catheter shaft 11. FIG. 1 illustrates the regrooming sheath 18 in the fully retracted configuration.

In the illustrated embodiment a finger hold section 19 at the proximal end of the regrooming sheath 18 is configured to facilitate the ability of the physician to grip the sheath 18. A releasable lock mechanism 32 configured to releasably lock the regrooming sheath 18 to the elongated shaft 11 is mounted on a proximal end section of the sheath 18. Although illustrated as a simplified structure at the proximal end of regrooming sheath 18 for clarity and ease of illustration, a more elaborate handle could be provided on the proximal end of the catheter system, which has a mechanism which can be activated to move the regrooming sheath 18 relative to the elongated shaft 11 therein, and which can have a lock to releasably secure the regrooming sheath 18 to the elongated shaft 11. Such handle mechanisms are generally known and typically include a thumb wheel, trigger, lever or other activation mechanism for advancing and/or retracting a shaft. A variety of suitable mechanisms may be used to clamp or otherwise releasably lock the regrooming sheath 18 to the elongated shaft 11 as are conventionally known, typically in the form of a clamp or other locking mechanism at or near the proximal end of the recovery sheath 18.

In the embodiment of FIG. 1, the balloon catheter shaft 11 comprises an inner tubular member 25 with the device lumen 13 therein, and an outer tubular member 26 with the inflation lumen 12 therein. As best shown in FIG. 2, illustrating a transverse cross section of the catheter 10 of FIG. 1 taken along line 2-2, the inner tubular member 25 extends coaxially within the outer tubular member 26 such that the inflation lumen 12 is the annular space therebetween. However, a variety of suitable balloon catheter shaft configurations can alternatively be used as are conventionally known, including dual lumen catheter shafts with side-by-side lumens. The balloon 15 has a proximal skirt section sealingly secured to the shaft outer tubular member 26 and a distal skirt section sealingly secured to the shaft inner tubular member 25, so that the interior of the balloon is in fluid communication with the inflation lumen 21.

FIG. 3 illustrates an enlarged longitudinal cross sectional view of the distal end of the balloon catheter of FIG. 1, taken within circle 3, and FIG. 4 is a transverse cross section of FIG. 3, taken along line 4-4. In the illustrated embodiment, the distal recovery tip 14 has a proximal end fixedly secured to the distal end of the inner tubular member 25 of the catheter shaft 11. The distal recovery tip 14 is typically fusion and/or adhesively bonded to the distal end of the inner tubular member 25. Alternatively, the distal recovery tip 14 can be formed as an integral, one-piece unit with the distal end of the inner tubular member 25. In a presently preferred embodiment, the distal recovery tip 14 is formed of a single layer of polymeric material which may be the same or different than polymeric material forming the inner tubular member 25 secured thereto. Although the inner tubular member 25 is illustrated as a single layer of polymeric material in FIG. 3, it should be understood that a variety of suitable catheter shaft configurations can be used including a multiple layered tubular member.

Typically, the inner diameter of the distal recovery tip 14 is larger than the inner diameter of at least the section of the inner tubular member 25 proximally adjacent thereto (e.g., the section of the inner tubular member 25 extending through the balloon inflatable interior to the distal tip 14). Similarly, the distal recovery tip 14 has an outer diameter which is larger than the outer diameter of at least the proximally adjacent section of the inner tubular member 25. In one embodiment, the wall thickness of the distal tip 14 is about equal to the wall thickness of the proximally adjacent section of the inner tubular member 25.

The length and diameter of the distal recovery tip 14 will vary depending upon the size and configuration of the expandable device to be recovered therein. In one embodiment, the distal recovery tip 14 has an inner diameter of about 0.02 to about 0.04 inches, and an outer diameter of about 0.03 to about 0.08 inches. Typically, the distal recovery tip 14 has a length of about 2 to about 100 mm, or more specifically, about 0.1% to about 15% of the total length of the balloon catheter shaft 11.

FIGS. 5-9 illustrate the balloon catheter 10 of FIG. 1 during a method in which the balloon catheter 10 is inflated to perform a medical procedure within a patient's body lumen 35 and then used to recover a radially expanded embolic protection device 40 previously deployed in the body lumen 35. Specifically, the balloon catheter 10 is advanced within the body lumen 35 to position the noninflated balloon 15 at a treatment site in the body lumen 35 and proximal to the distal end of the deployed embolic protection device 40 (see, e.g., FIG. 5). The embolic protection device 40 is of the type having a self-expanding frame 41 on a distal section of an elongated core wire 42, and FIG. 5 illustrates the device 40 with the frame radially expanded into contact with the vessel wall inner surface such that the frame will filter or trap embolic material in the body lumen 35. Typically, the embolic protection device 40 is delivered and deployed in the body lumen 35 using a delivery catheter (not shown) which is then removed prior to positioning of the balloon catheter 10.

In the embodiment illustrated in FIG. 5, the balloon 15 is positioned within a stent 50 which requires a post-dilation (stent touch-up) procedure, commonly performed on self-expanding stents in order to radially expand the stent against the inner surface of the vessel wall to a fully expanded configuration. Thus, the stent 50 has been previously delivered and deployed within the body lumen 35 using a stent delivery catheter (not shown) which is then removed prior to positioning of the balloon catheter 10. Following removal of the stent delivery catheter, the balloon catheter 10 of the invention is introduced into the body lumen 35 and slidably advanced to the treatment site in the low profile noninflated configuration, over the previously deployed embolic protection device 40. Specifically, the device lumen 13 of the balloon catheter shaft inner tubular member 25 is configured to slidably receive and track over the core wire 42 of the embolic protection device 40.

The balloon 15 is inflated in the body lumen 35 to perform a medical procedure, which in the illustrated embodiment is a post-dilation of the self-expanded stent 50. FIG. 6 illustrates the balloon 15 inflated within the stent 50 in order to radially expand the stent 50 to a fully expanded configuration to thereby implant the stent in the body lumen 35, with the embolic protection device 40 remaining deployed distal to the stent 50 to capture any embolic material released during the procedure. The balloon 15, configured for radially expanding stent 50, typically has a relatively high working pressure (for example, a nominal pressure of about 6 to about 12 atm), and a relatively high wall strength, to expand the stent without rupturing.

During inflation of the balloon 15, the distal end of the recovery sheath 18 on the catheter shaft 11 is positioned proximal to the balloon 15. After being inflated, the balloon 15 is deflated to a deflated configuration having wrinkles and folds or wings of excess balloon material. Prior to being regroomed the deflated balloon 15 therefore has a larger profile than the noninflated balloon (in the low profile configuration of FIG. 1). FIG. 7 illustrates the balloon catheter 10 after the inflated balloon of FIG. 6 has been deflated. In accordance with the invention, the deflated balloon is then regroomed by slidably displacing the regrooming sheath 18 and balloon catheter balloon 15 relative to one another to position the balloon 15 within the regrooming sheath 18, to thereby reduce the profile of the deflated balloon 15 to a regroomed configuration. Typically, the regrooming sheath 18 is distally advanced over the deflated balloon 15. Although the deflated balloon can alternatively be proximally withdrawn into the regrooming sheath to regroom the balloon, this is much less preferred due to the risk that the deflated balloon will snag or otherwise disadvantageously interact with the stent or anatomy. Thus, the method preferably involves regrooming the balloon without longitudinally displacing the balloon until the regrooming sheath is surrounding at least a portion of the inflatable length of the balloon. FIG. 8 illustrates the balloon catheter during regrooming, with the regrooming sheath distally advanced over the deflated balloon within the stent 50, such that the balloon 15 is regroomed at the treatment site in the body lumen 35.

The small inner diameter of the regrooming sheath collapses and presses the deflated balloon material to a lower profile. Typically, the balloon 15 and regrooming sheath 18 are rotated relative to one another by, for example, torquing the balloon catheter shaft 11 proximal end to rotate the balloon within the regrooming sheath 18, to thereby wrap the pressed balloon material around the catheter inner tubular member 25.

In accordance with the invention, with the balloon in the regroomed configuration, the expanded embolic protection device frame 41 is then collapsed by distally advancing the balloon catheter shaft 11, to advanced the distal recovery tip 14 over at least a portion of the expanded section of the embolic protection device 40. The inner surface of the distal recovery tip 14 contacts a portion of the expanded frame 41 or a collapsing mechanism connected thereto such as control wires or other mechanisms as are conventionally known for embolic protection filters, thereby collapsing the frame 41 as the distal recovery tip is advanced distally. As a result, the regroomed balloon 15 is advanced distally of the regrooming sheath 18 and stent 50. Although illustrated in the embodiment of FIG. 9 with the entire length of the expandable frame 41 of the embolic protection device 40 located within the distal recovery tip 14, in alternative embodiments the embolic protection device design can be configured to fully collapse with less of the length of the expandable section positioned within the distal recovery tip 14. Following recovery of the device 40, the regroomed balloon catheter 10 with the frame 41 in the collapsed configuration therein, is slidably displaced in the patient's body lumen 35 to reposition or remove the frame 41 from the patient's body lumen 35.

In one embodiment, the distal end section of the regrooming sheath 18 and has a wall thickness less than the wall thickness of the distal recovery tip 14. In one embodiment, the distal end section of the regrooming sheath 18 has an inner diameter less than the inner diameter of the section of the device lumen 13 within the distal tip 14. As a result, the regrooming sheath is configured to regroom the balloon but is not configured for advancement over the distal recovery tip 14 of the catheter. The relatively small size of the inner diameter of the regrooming sheath provides for low profile, tight regrooming of the balloon. However, due to the small inner diameter of the distal end section of the regrooming sheath 18, forcing the regrooming sheath distally over the embolic protection device frame 41 could break the connection between the frame 41 and the elongated core 42 of the device 40. Thus, the regrooming sheath 18 is not configured for recovery of the expandable device 40. For example, in one embodiment, the embolic protection device 40 or other recoverable expandable device has an elongated body which has the expanded frame secured to a distal section thereof with a detach force of less than 1 pound, and the regrooming sheath has an inner diameter configured to fit tightly on the deflated balloon but not configured to accommodate or collapse the frame (e.g., the expanded frame cannot be slid within the regrooming sheath to collapse the frame without a force exceeding the detach force of the frame).

In one embodiment, the inner diameter of at least a distal section of the regrooming sheath 18 is less than or about equal to the outer diameter of the noninflated balloon. As a result, the regrooming sheath 18 is preferably positioned proximal to the noninflated balloon during advancement of the catheter 10 prior to inflation of the balloon (see, e.g., FIG. 5). In one embodiment, at least the distal section of the regrooming sheath has a relatively low strength (small wall thickness and/or low durometer stiffness material) to maintain the flexibility of the distal section of catheter 10. The sheath 18 is therefore not typically configured to prevent the balloon from inflating, in that the wall of the regrooming sheath (if advanced over the noninflated balloon prior to inflation of the balloon) would typically yield to the radially expansive force of the inflating balloon at the relatively high working pressures of the balloon.

The diameter of the regrooming sheath 18 depends upon the size of the balloon 15 of the catheter 10. Typically the regrooming sheath has an inner diameter of about 0.04 to about 0.09 inches, and an outer diameter of about 0.05 to about 0.10 inches. Although the regrooming sheath 18 illustrated in the figures has a constant inner and outer diameter along the entire length thereof, in alternative embodiments (not shown) the regrooming sheath 18 is profiled as for example with an inner diameter which steps or tapers down to a smaller inner diameter along a distal end section of the sheath 18.

In one embodiment, the regrooming sheath 18 is configured for removal from the balloon catheter shaft 11. For example, the regrooming sheath 18 can be provided with a weakened wall portion (not shown) configured to allow for peeling or tearing the sheath 18 off the shaft 11, so that the regrooming sheath 18 can be removed independently of the balloon catheter shaft 11 following regrooming of the balloon 15. Alternatively, the regrooming sheath 18 remains on the shaft 11 during recovery of the embolic protection device 40 and subsequent removal of the balloon catheter 10 (with the collapsed device 40 therein) from the body lumen 35.

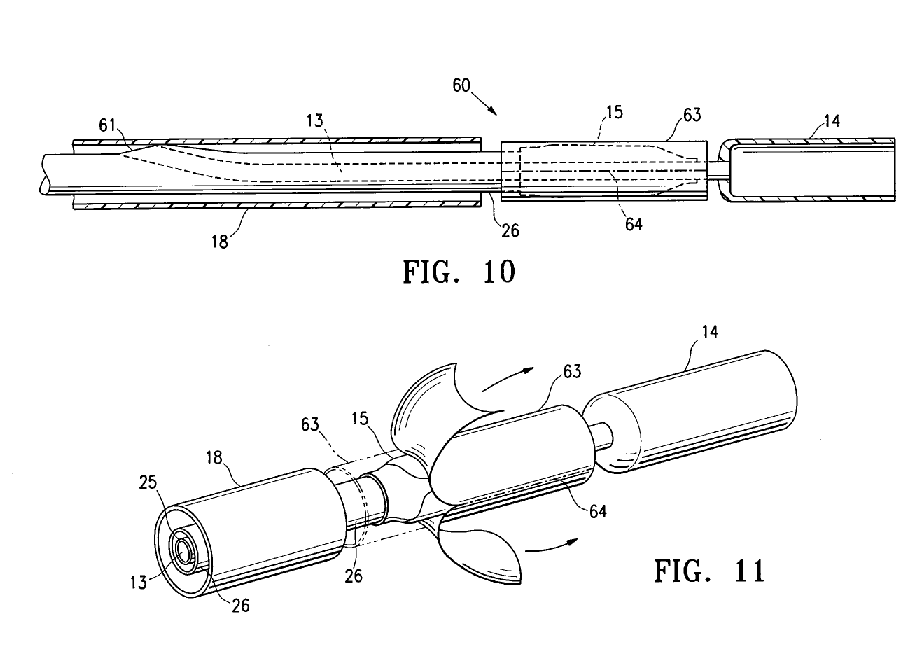

Although the balloon catheter 10 in the embodiment of FIG. 1 is an over-the-wire type catheter having device (e.g., wire-receiving) lumen 13 extending from the proximal to the distal end of the balloon catheter 10, the balloon catheter of the invention can alternatively be a rapid exchange type catheter in which the device lumen 13 extends to a proximal port spaced distally from the proximal end of the catheter 10. FIG. 10 illustrates an embodiment of a rapid exchange type balloon catheter 60, otherwise similar to the embodiment of FIG. 1 but having a relatively short device lumen 13 (shown in dashed line in FIG. 10) extending to a rapid exchange proximal port 61 spaced distally from the proximal end of the shaft 11. The catheter 60 is advanceable over the embolic protection device 40 (not illustrated in FIG. 10) to perform a procedure in accordance with the invention as discussed above in relation to the embodiment of FIG. 1, but the core wire 42 of the embolic protection device 40 exits the catheter 60 at the rapid exchange proximal port 61.

In the embodiment of FIG. 10, the balloon catheter 60 has a protective cover 63 mounted on the balloon 15, which is removed from the balloon 15 and discarded prior to use of the catheter 60. Specifically, the cover 63 has an inner diameter which is sized to slidably receive the balloon therein and to frictionally fit on the noninflated balloon 15 (preferably without heat shrinking the cover 63). The inner diameter of the cover 63 is smaller than both the outer diameter of the distal recovery tip 14 of the balloon catheter and at least a section of the catheter shaft 11 proximal to the balloon 15, and is therefore positioned on the wrapped noninflated balloon 15 prior to attachment of the distal recovery tip 14 on the distal end of the catheter 60. The cover 63 has a weakened wall portion configured for separating, to facilitate removal of the cover from the balloon catheter 60 prior to use of the balloon catheter 60, by peeling the cover 63 from the balloon. A variety of suitable weakened wall portions can be used including one or more notched, scored, or perforated lines in the wall of the cover. FIG. 11 is a perspective view of the distal end of the balloon catheter 60 of FIG. 10, during peeling away of the balloon protective cover 63 prior to use of the catheter 60. In the illustrated embodiment, the weakened wall portion comprises a first score line 64 and a second score line (not shown) on the opposite side of the cover, extending the length of the cover 63. In one embodiment, a notch (not shown) at one end of the cover cuts through the wall of the cover at a location in line with the score line 64, to provide a starting point for the physician to part the sheath along the score line(s) 64. Although the illustrated cover 63 has two score lines 64 which part the cover into two halves, in an alternative embodiment the cover has a single score line 64. In another alternative embodiment (not shown), the cover 63 has two score lines adjacent to one another on one side of the cover, each extending the length of the cover 63 to a tab at the end of the cover 63, such that the tab can be pulled to separate a thin strip of the cover between the two adjacent score lines. The cover 63 is typically formed of a polymeric material such as linear low density polyethylene (LLDPE), or LDPE, HDPE, polyether block amide (PEBAX), silicone, latex, or TEFLON, and is preferably a relatively elastic material to facilitate tearing of the cover 63 during removal from the balloon 15.

A variety of suitable conventional balloon materials and formation methods can be used to form the balloon 15 of catheter 10/60, including polyamides such as nylon, copolyamides such as PEBAX, polyurethanes, polyolefins, PET, and the like. The balloon is typically formed by blow-molding as is conventionally known, which shapes and defines the nominal inflated diameter of the balloon in the working pressure range, and the noninflated configuration typically has tightly wrapped wings formed by applying heat and a radially collapsing force to the balloon to set the balloon in the low profile noninflated configuration. Although the illustrated balloon 15 has wings wrapped therearound in the noninflated configuration, the balloon can have a variety of suitable noninflated configurations as are conventionally known. However, the balloon catheter of the invention will have a balloon which deflates to a relatively high profile due to the wrinkles or folds of deflated balloon material. Therefore, the balloon is unlike highly elastic balloons which elastically recoil from the inflated profile back down to the noninflated low profile configuration upon deflation.

The regrooming sheath 18 can be formed of a variety of suitable materials commonly used in catheter shaft construction including thermoplastic elastomers or thermoset plastics. For example, in one embodiment, the regrooming sheath 18 is formed at least in part of a polyamide copolymer (a thermoplastic elastomer) such as a PEBAX. In one embodiment, the regrooming sheath is a single lumen tube having a polymeric wall with a wall thickness of not greater than about 0.004 to about 0.040 inches. The wall of the sheath 18 may include a lubricity enhancing additive or coating. The distal recovery tip 14 of the balloon catheter 10/60 can similarly be made from a variety of suitable materials having sufficient strength to hold the compressed strut assembly of the embolic protection device 40, such as a cross-linked HDPE or other polyolefins, preferably having a relatively lubricious, low friction surface to minimize friction between the filtering assembly and the distal recovery tip 14 inner surface. In one embodiment, a lubricious surface coating, such as a silicone lubricant, is provided on the inside surface of the distal recovery tip 14 to further reduce the frictional force during contact with the embolic protection device frame 41.

The dimensions of balloon catheter 10/60 are determined largely by the size of the balloon and guidewire to be employed, the catheter type, and the size of the artery or other body lumen through which the catheter must pass or the size of the stent. The overall length of the catheter 10/60 may range from about 100 to about 150 cm, and is typically about 143 cm. The length of the regrooming sheath 18 will depend on the size of the balloon catheter shaft 11 and balloon 15, and is generally about 55 to about 110 cm. Typically, the outer tubular member 26 has an outer diameter of about 0.025 to about 0.04 inch (0.064 to 0.10 cm), usually about 0.037 inch (0.094 cm), and the wall thickness of the outer tubular member 26 can vary from about 0.002 to about 0.008 inch (0.0051 to 0.02 cm), typically about 0.003 to 0.005 inch (0.0076 to 0.013 cm). The inner tubular member 25 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). Preferably, balloon 15 has a length about 0.8 cm to about 6 cm, and an inflated working diameter of about 2 mm to about 10 mm.

While the present invention is described herein in terms of certain preferred embodiments, those skilled in the art will recognize that various modifications and improvements may be made to the invention without departing from the scope thereof. For example, although discussed primarily in terms of recovery of an embolic protection filter having a frame of space apart, longitudinal struts, alternative recoverable expandable devices can be recovered using the catheter system 10/60 including embolic protection devices not having this frame-type construction, and expanded agent/drug delivery devices, and the like. Additionally, although the shaft is illustrated as having an inner and outer tubular member, a variety of suitable shaft configurations may be used including a dual lumen extruded shaft having a side-by-side lumens extruded therein. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments of the invention.

We claim:

1. A method of using a balloon catheter to perform a medical procedure and recover an expanded frame of a expandable device in a patient's body lumen, comprising:
a) advancing within the patient's body lumen a balloon catheter having an elongated shaft with an inflation lumen, an inflatable balloon secured to a distal shaft section such that an interior of the balloon is in fluid communication with the inflation lumen, a distal tip configured to slidably receive at least a portion of the expandable device to collapse the expanded frame to a collapsed configuration, and a regrooming sheath releasably locked to the elongated shaft and slidably disposed on the shaft in an unlocked configuration;
b) inflating the balloon in the patient's body lumen at a treatment location longitudinally adjacent to the expanded frame to perform a medical procedure, deflating the balloon, and regrooming the deflated balloon by slidably displacing the regrooming sheath and balloon relative to one another to position the balloon within the regrooming sheath, thereby reducing the profile of the deflated balloon to a regroomed configuration;
c) collapsing the expanded frame by sliding the balloon catheter out of a distal end of the regrooming sheath, such that the balloon in the regroomed configuration is positioned distal to the regrooming sheath and the distal tip of the balloon catheter is advanced over at least a portion of the expandable device; and
d) slidably displacing the balloon catheter, with the expandable device in the collapsed configuration therein, to reposition or remove the frame from the patient's body lumen.

2. The method of claim 1 wherein the balloon is inflated to radially expand a stent in the body lumen, and the balloon is regroomed in (b) by distally advancing the regrooming catheter over the deflated balloon within the radially expanded stent.

3. The method of claim 1 wherein the balloon catheter is slidably advanced in the body lumen with the regrooming sheath releasably locked thereto, and with the distal end of the regrooming sheath located proximal to the balloon, to position the balloon at the treatment location before (b).

4. The method of claim 1 including releasably locking the regrooming sheath to the balloon catheter after (c) and before (d), with the distal end of the regrooming sheath located proximal to the balloon.

5. The method of claim 1 wherein the expanded frame is a self-expanding embolic protection device frame, and collapsing the frame in (c) comprises positioning the distal end of an expandable portion of the frame within the balloon catheter distal tip such that the frame is collapsed to a fully collapsed configuration for removal from the body lumen by the balloon catheter distal tip.

6. The method of claim 1 wherein regrooming the balloon includes rotating the balloon relative to the regrooming sheath.

7. The method of claim 1 wherein the expandable device has an elongated body which has the expanded frame secured to a distal section thereof with a detach force of less than 1 pound, and the regrooming sheath has an inner diameter configured to fit tightly on the deflated balloon, such that the expanded frame cannot be slid within the regrooming sheath to collapse the frame without a force exceeding the detach force of the frame.

8. The method of claim 1 including before a), peeling a protective tubular cover off of the balloon, the cover having an inner diameter which is sized slidably receive the balloon therein and to frictionally fit on the noninflated balloon and which is smaller than both the outer diameter of the distal tip of the balloon catheter and at least a section of the catheter shaft proximal to the balloon, and having a weakened wall portion configured for separating to peel the cover from the balloon for removal of the cover from the balloon catheter prior to use of the balloon catheter.

9. A method of using a balloon catheter to perform a medical procedure and recover an expanded embolic protection device in a patient's body lumen, comprising:
a) advancing within the patient's body lumen a balloon catheter having an elongated shaft with an inflation lumen, an inflatable balloon on a distal shaft section with an interior of the balloon is in fluid communication with the inflation lumen, a distal tip configured to slidably receive at least a portion of an expanded section of the embolic protection device to collapse the expanded section to a collapsed configuration, and a regrooming sheath releasably locked on the elongated shaft and slidably disposed thereon in an unlocked configuration;
b) inflating the balloon from a noninflated configuration to an inflated configuration to radially expand a stent in the patient's body lumen at a treatment location longitudinally adjacent to the expanded section of the embolic protection device, and deflating the balloon to a deflated configuration, the deflated balloon having folds of excess material, and regrooming the deflated balloon by slidably displacing the regrooming sheath and balloon relative to one another to position the balloon within the regrooming sheath, to reduce the profile of the deflated balloon to a regroomed configuration;
c) collapsing the expanded embolic protection device by sliding the balloon catheter out of a distal end of the regrooming sheath, such that the distal tip of the balloon catheter is advanced over at least a portion of the expanded section of the embolic protection device, and such that the balloon in the regroomed configuration is positioned distal to the regrooming sheath and distal to the stent; and
d) withdrawing the balloon catheter, with the embolic protection device in the collapsed configuration therein, proximally through the stent, to reposition or remove the embolic protection device from the patient's body lumen, such that an outer surface of the deflated balloon in the regroomed configuration is exposed to but radially spaced from the stent as the balloon catheter is withdrawn through the stent.

10. The method of claim 9 wherein the stent is a self-expanding stent at least partially expanded against an inner surface of the body lumen wall before (b), and including before (b) slidably advancing the balloon within the stent, so that inflating the balloon radially expands the stent to a fully expanded configuration, to implant the stent in the body lumen.

11. The method of claim 9 wherein the stent is a balloon expandable stent mounted on the balloon before (a), so that inflating the balloon radially expands the stent against an inner surface of the body lumen wall to implant the stent in the body lumen.

12. The method of claim 9 wherein the distal tip of the balloon catheter has a length sufficiently short so that the distal end of the distal tip is proximal to the expanded section of the embolic protection device during inflation of the balloon in (b).

13. The method of claim 9 wherein the regrooming sheath and the balloon catheter are advanced together in the locked configuration with the distal end of the regrooming sheath positioned proximal to an inflatable section of the balloon in (a).

14. A balloon catheter configured for recovery of an expanded device from within a patient's body lumen, comprising:
a) an elongated shaft with an inflation lumen and a device lumen, and a distal tip which defines a distal end of the device lumen and which is configured to slidably receive the expanded device therein to collapse the device to a collapsed configuration, wherein the device lumen inner diameter increases at the distal tip;
b) an inflatable balloon on a distal shaft section, having an interior in fluid communication with the inflation lumen such that the balloon has an inflatable section; and
c) a regrooming sheath releasably locked on the elongated shaft and slidably disposed thereon in an unlocked configuration, comprising a tubular body with a lumen, the lumen having at least a distal end section which has an inner diameter less than a transverse dimension of the deflated balloon and less than an outer diameter of the distal tip, and which is configured to slidably receive the deflated balloon therein to reduce the profile of the deflated balloon to a regroomed configuration, and the regrooming sheath having a length less than the balloon catheter shaft such that in a fully retracted configuration the distal end of the regrooming sheath is proximal to the inflatable section of the balloon.

15. The catheter of claim 14 wherein the inner diameter of the distal end section of the regrooming sheath is less than the inner diameter of the section of the device lumen within the distal tip.

16. The catheter of claim 14 wherein the inner diameter of the distal section of the regrooming sheath extending fully around the circumference thereof is less than or about equal to the outer diameter of the noninflated balloon.

17. The catheter of claim 14 wherein the distal tip of the balloon catheter has a length of less than about 25 mm.

18. The catheter of claim 14 wherein the regrooming sheath is a single lumen tube having a polymeric wall with a wall thickness of not greater than about 0.004 to about 0.040 inches.

19. The catheter of claim 14 wherein the device lumen extends from a distal port in the distal end of the distal tip to a proximal port spaced distally from the proximal end of the shaft.

20. The catheter assembly of claim 19 including a protective tubular cover on the balloon, having an inner diameter which is sized slidably receive the balloon therein and to frictionally fit on the noninflated balloon and which is smaller than both the outer diameter of the distal tip of the balloon catheter and at least a section of the catheter shaft proximal to the balloon, and having a weakened wall portion configured for separating to peel the cover from the balloon for removal of the cover from the balloon catheter prior to use of the balloon catheter.
